(19) United States
(12) Patent Application Publication
(10) Pub. No.: US 2020/0168292 A1
(43) Pub. Date: May 28, 2020

(54) IDENTIFICATION OF ENZYME-SUBSTRATES ASSOCIATIONS

(71) Applicant: CASE WESTERN RESERVE UNIVERSITY, Cleveland, OH (US)

(72) Inventors: Mark A. Chance, Cleveland, OH (US); Marzieh Ayati, Cleveland, OH (US); Mehmet Koyuturk, Cleveland, OH (US)

(21) Appl. No.: 16/618,733

(22) PCT Filed: Jun. 4, 2018
(86) PCT No.: PCT/US18/35884

§ 371 Date: Dec. 2, 2019

Related U.S. Application Data
(60) Provisional application No. 62/514,511, filed on Jun. 2, 2017.

(51) Int. Cl. G16B 20/20; G16B 40/10; G06N 5/04

(52) U.S. Cl. CPC G16B 20/20; G06N 5/04; G16B 40/10

(57) ABSTRACT

A method includes receiving proteomic data for each of a plurality of identified post-translational modification (PTM) sites. A pairwise correlation of co PTM is computed among each respective pair of the identified PTM sites. The method also includes generating a co-PTM network based on each pairwise correlation to describe co PTM characteristics for the identified PTM sites. The method also includes predicting enzyme-substrate associations for each of the plurality of identified PTM sites based on the co-PTM network and a set of predetermined enzyme-substrate associations.

### CROSS REFERENCE TO RELATED APPLICATION

This application claims the benefit of U.S. Provisional Patent Application No. 62/514,511, filed Jun. 2, 2017, and entitled PREDICTING KINASE-SUBSTRATE INTERACTIONS USING PATTERNS OF CO-PHOSPHORYLATION, which is incorporated herein by reference in its entirety.

### GOVERNMENT INTEREST

This invention was made with government support under Grant Nos. R01-GM11720801 and P30CA043703 awarded by the United States National Institute of Health (NIH). The United States government has certain rights to the invention.

### TECHNICAL FIELD

This disclosure relates to identifying enzyme-substrate associations.

### BACKGROUND

Protein phosphorylation (PP) is a post-translational modification that is central to cellular signaling where networks composed of kinases, phosphatases, and their substrates regulate the sites and levels of phosphorylation at the molecular level. Kinases play an important role to increase the levels of protein based phosphorylation critical for cellular regulation while phosphatases decrease the phosphorylation levels; both kinases and phosphatases have emerged as an important class of drug targets for many diseases, particularly cancers, as their inhibition can reduce (kinase inhibitors or phosphatase activators) or increase (kinase activators or phosphatase inhibitors) cellular phosphorylation levels. Despite significant success in identifying cellular kinase (and phosphatase) substrates, the identity of the responsible kinases (or phosphatases) is typically unknown for >90% of the cases, so drug targeting to modulate phosphorylation or dephosphorylation of key regulatory sites based on these data are inefficient. For this reason, effective bioinformatics tools to predict KSAs (or the corresponding phosphatase substrate associations) have been developed to fill the gap between phosphosite (substrate) identification and kinase (or phosphatase) annotation. Examples of databases listing known KSAs include PhosphoSitePlus (available from Cell Signaling Technology, Inc. of Danvers, Mass. Examples of a tool for predicting KSAs include KinomeXplorer (disclosed at Horn H, et al. “KinomeXplorer: an integrated platform for kinome biology studies”, Nat. Methods, vol. 11, pg. 603-604 (May 2014)). Kinome explorer and other existing computational tools for prediction of kinase-substrate associations rely on static information, such as sequence motifs and physical interaction. Accordingly, more comprehensive identification of links between kinases and their substrates can enhance the ability to understand the underlying mechanism of diseases and signaling networks to drive drug discovery.

### SUMMARY

As one example, a system includes one or more non-transitory machine readable media to store instructions executable by a processor to perform a method. The instructions include correlation calculator code to compute a co-post-translational modification (PTM) value based on a measure of correlation of intensity data for pairs of PTM sites identified in proteomic data. The instructions also include co-PTM network generator code to construct a co-PTM network for the plurality of identified PTM sites based on the co-PTM values. The instructions also include predictor code to identify enzyme-substrate associations based on analyzing the co-PTM network with respect to a set of predetermined substrate associations.

As another example, a method includes receiving proteomic data representing intensity values for each of a plurality of identified of post-translational modification (PTM) sites. A pairwise correlation of co-PTM is computed among each respective pair of the identified PTM sites. The method also includes generating a co-PTM network based on each pairwise correlation to describe co-PTM characteristics for the identified PTM sites. The method also includes predicting enzyme-substrate associations for each of the plurality of identified PTM sites based on the co-PTM network and a set of predetermined enzyme-substrate associations.

### BRIEF DESCRIPTION OF THE DRAWINGS

FIG. 1 depicts an example of a system to identify kinase-substrate associations.

FIG. 2 depicts an example of a co-phosphorylation network.

FIG. 3 an example of a co-phosphorylation network showing predicted kinase-substrate associations.

FIG. 4 is an example of another system to identify kinase-substrate associations.

FIGS. 5A and 5B are graphs demonstrating example distributions of co-phosphorylation between phosphosites that are substrates of the same kinase.

FIG. 6 depicts an example of a protein-protein interaction network of physically interacting proteins with subnetworks associated with different pathways.

FIG. 7 is a flow diagram depicting an example method to identify kinase-substrate associations.

FIG. 8 is a flow diagram depicting another example method to identify kinase-substrate associations.

FIGS. 9A and 9B are graphs demonstrating comparative rankings of kinases identified by different example methods.

FIGS. 10A and 10B depict examples of box plot distribution of the rank of the target kinase determined according to different example methods.

FIG. 11 is a table demonstrating a comparison of reported kinases and predicted kinases determined according to different example methods.

### DETAILED DESCRIPTION

This disclosure relates to identification of enzyme-substrate associations ESAs. The enzyme-substrate associations thus can be determined to specify any enzymes that make post translational modifications (PTMs) (e.g., adding or removing) a functional chemical group (e.g., phosphate group, acetyl group, acetate group, oxidation, or other specific chemical modification, etc.) with respect to a biomolecule such as including proteins, lipids or carbohydrates. The patterns of intensities (co-modification pattern) for such functional chemical groups, as revealed by examination of a sufficient number of biological states, can be analyzed to reveal unknown ESAs based on a set of known signatures of a relatively small set of available ESAs. In some examples, as described herein, the ESAs may include kinase-substrate associations (KSAs) for phosphorylation and/or phosphatase-substrate associations (PSAs) for dephosphorylation phosphosites detected based on mass spectrometry (MS) or other dynamic data acquired on a range of biological samples of interest.

As an example, MS data is determined describes intensity data for each of a plurality of identified peptides corresponding to phosphorylation sites (also referred to herein, e.g., as phosphopeptide substrates, or phosphosites). The MS data for different samples can thus provide information that can capture the spatio-temporal dynamics of phosphorylation events, based on intensity changes across a series of biological samples, where the samples represent a reference state of interest (e.g. tumor type or specifically identified or classified patient or animal samples). A co-phosphorylation network is constructed based on pairwise correlation of the intensity data for the peptides identified in the MS data across all selected samples. As used herein, co-phosphorylation refers to a measure of similarity between a pair of phosphosites; for example, describing how similar a pattern of phosphorylation is for the pair of phosphosites across different biological states represented by the choice of experimental samples. Statistically significant co-phosphorylation values (e.g. those of high similarity) are verified by permutation analysis (switching the labels of the phosphosites or samples), e.g. the phosphorylation distribution is wider for the un-permuted distribution. KSAs and/or PSAs can be predicted based on the co-phosphorylation network and a set of predetermined substrate associations. For example, a Bayesian method can be applied to transfer associations from the predetermined substrate associations to phosphosites in the network.

In some examples, the context-specific ESAs can be combined with another set of ESAs (e.g., static ESA information), such as derived from analysis of sequence and protein interaction data, to provide further accurate predictions of ESAs where such static ESA information is available. The approach disclosed herein directly incorporates the dynamics of PTM into ESA prediction methods. Since cellular signaling is a highly dynamic and transient process, the incorporation of specific information on the dynamics of PTMs into ESA predictions (e.g., via the MS data), thus should enable “global” scale predictions (e.g. for all of the MS data).

Additionally, as some functional chemical groups (e.g., phosphosites) can be modified by multiple catalyzing enzymes and further depending on its various biological context, ascertaining which individual enzyme is activated in different cell or tissues types is challenging to predict and yet often crucial to the drug discovery process. However, in many existing bioinformatic tools, such as those relying on sequence motifs, structural information, and protein interactions, ESA predictions are unable to represent a specific biological context, at least with the current state of cell based information. Therefore, an important benefit of integrating context specific data (e.g., MS data or similar data) into ESA predictions is that, as disclosed herein, the prediction data can capture the context-specificity of these interactions.

The systems and methods disclosed herein thus can provide critical insights into biological processes in both development and disease. As one example, by identifying a greater number of kinase-substrate associations and/or phosphatase-substrate associations, the systems and methods disclosed herein can provide global predictions of KSAs and/or PSAs, which may lead to development of kinase inhibition and/or activation strategies for combating multiple cancer related phenotypes. While many examples disclosed herein are in reference to predicting KSAs, the approach herein is equally applicable to predicting other ESAs, including PSAs.

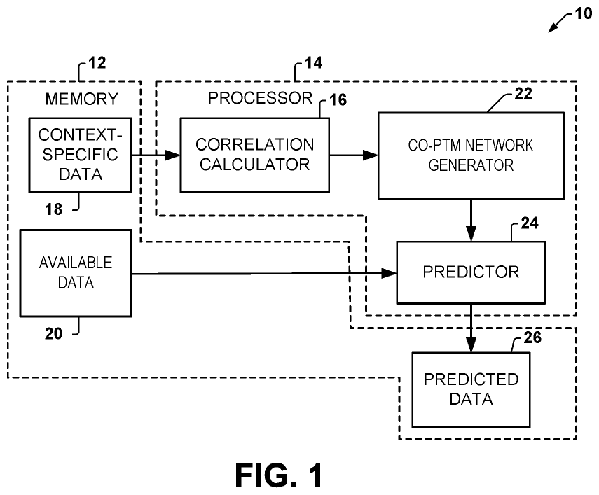

FIG. 1 depicts an example of a system to predict ESAs. In the example of FIG. 1, the system 10 includes memory 12 and one or more processors 14. The memory 12 can be implemented to include volatile and/or non volatile memory which may be local or remote or be distributed among local and remote storage devices. For example, the memory may reside locally or connected to the processor through the corresponding bus. In other examples, the memory may include a remote memory structure (e.g., a storage area network and/or cloud resource) readily accessible via a communication link. The memory stores data 18, 20 and 26 and is accessible by the processor 14, which may include one or more processor cores. For example, the processor 14 can access the memory 12 to receive to machine-executable instructions (e.g., code) that is executable by the processor to perform functions or methods disclosed herein. A user interface (not shown) may be utilized to specify (e.g., a file or other resource location) for data that is to be utilized by the processor.

In the example of FIG. 1, the processor 14 includes a correlation calculator 16 that computes a measure of correlation among peptides specified in context specific data 18. As one example, the context specific data 18 may be implemented as mass spectrometry (MS) data. For example the MS data 18 includes intensity values (e.g., phosphorylation levels) for identified peptides from one or more samples. Samples can be tissue samples, cell lines or other replicated samples that reflect a cross section of the biological state of interest. In another example, the context specific data 18 may be determined from antibody arrays designed for detecting phosphopeptide and phosphoprotein sites that provide corresponding PTM intensity data. The correlation calculation 16 thus provides code executable by the processor 14 to compute a measure of correlation of intensity data for each pair of PTM sites (e.g., functional groups identified in the data to undergo PTM) identified in the data 18. In an example using MS data 18, the correlation calculator 16 can compute a measure of correlation of intensity data for each pair of phosphorylation sites (also referred to as phosphosites) such as identified in MS data.

In some examples, the context-specific data 18 (e.g., MS data) includes a PTM profile (e.g., phosphorylation profile) for each PTM site (e.g., phosphosite) that is identified based on the context specific data. In this way, the context-specific data 18 can represent a number of biological states (e.g., originating from separate independent samples). Stated differently, the number biological states can define the number of dimensions in the PTM profiles of PTM sites in the input data 18. In some examples, the PTM profiles are provided as phosphorylation profiles describing the respective biological states. The number of biological states is to be sufficient to provide statistically significant co-PTM measures among the PTM profiles. In one example, the number of biological states include five states or, in another example, more than five biological states, such as six or more biological states. For example, the number of biological states corresponds to the number of independent samples (e.g., tumors) in the data 18. Examples of biological states include different dosage, different tumors, different samples of the same tumor, samples taken at different time points to name a few. Thus, the biological states may vary depending on the experiment being conducted.

The correlation calculator 16 thus computes co-PTM among each PTM site identified in the context-specific data 18. As one example, the correlation calculator 16 is programmed to compute a biweight midcorrelation according to the phosphorylation profile of each pair of phosphosites identified in the MS data 18. As a further example, the biweight midcorrelation of two vectors (e.g., phosphosite vectors) x∈R^{1×m} and y∈R^{1×m} is computed as

$${c}_{xy} = {\sum }_{i = 1}^{m} {x}_{i}^{′} {y}_{i}^{′}$$

$$where , \\ {x}_{i}^{′} = \frac{( {x}_{i} - med ( x ) ) {w}_{i}^{x}}{\sqrt{{{\sum }_{j = 1}^{m} [ {x}_{i} - med ( x ) ) {w}_{i}^{2} ]}^{⋀} 2}} , \\ {w}_{i}^{x} = {( 1 - {\vert \frac{{x}_{i} - med ( x )}{9 mad ( x )} \vert}^{2} )}^{2} I ( 1 - \vert \frac{{x}_{i} - med ( x )}{9 mad ( x )} \vert ) .$$

med(x) represents the median of vector x and mad(x) represents the median absolute deviation of vector x. I(u) is the indicator function which takes on value 1 if u>0 and 0 otherwise. y′ᵢ and wᵢ^{y} are also computed similarly for vector y.

Other forms of correlation (e.g., Pearson's correlation) or other methods to measure the correlation between PTM profiles of respective phosphosite pairs may be used in other examples.

A co-PTM network generator 22 is programmed to generate a co-PTM network based on the correlation data (e.g., determined by calculator 16). As an example, the network generator 22 is programmed to produce a co-phosphorylation network based on the correlation data (e.g., data describing phosphorylation levels among phosphosites). The network, which may be stored in memory as a graph data structure that includes nodes representing each of the identified PTM sites (and corresponding PTM profiles) in the data 18 and an edge between each respective pair of the nodes. Each edge is weighted based on a co-PTM value, as computed by correlation calculator 16, for the pair of corresponding PTM sites at nodes between which each edge extends. For example, for each pair of phosphosites p, q∈P, where P denotes the set of all phosphorylation sites that are present in the data 18, the co-phosphorylation of p and q as c_{pq} which can be determined as a correlation (e.g., the biweigth midcorrelation) as disclosed herein.

The processor 14 also executes a predictor code 24 to generate predicted data 26 based on the co-phosphorylation network and available data 20. The available data 20 provides a set of predetermined ESAs (e.g., KSAs or PSAs), such as may be stored in database. As disclosed herein the predicted data 26 may include predicted KSAs, predicted PSAs or a combination of KSAs and PSAs. The resulting predicted data 26 thus provides a probability (e.g., a likelihood value) for substrate interactions for each of the identified phosphosites. For example, the predictor can implement a Naïve Bayes framework to transfer annotations from the known available (e.g., KSA and/or PSA) data 20 to the phosphosites (nodes) in the co-phosphorylation network.

By way of further example, co-phosphorylation of two phosphosites can be computed only if both phosphosites are present in the MS data 18 (both sites are in P). Let A denote the distribution of co-phosphorylation among all pairs of phosphosites in P (i.e., A is the set of co-phosphorylation values c_{pq} across all (p, q) ∈P×P). On the other hand, kinase information is available for the phosphosites that are in T, where T denotes a set of all phosphorylation sites in the available association data 20. In this example, two phosphosites are a shared-kinase pair if the two phosphosites are known to be regulated by the same kinase. For example, the regulation of phosphosites by a given known kinase may be predetermined and stored in one or more databases (e.g., corresponding to available data 20), such as including PhosphoSitePlus or The human DEPhOsphorylation database (DEPOD). As an example, the distribution of co-phosphorylation among shared-kinase pairs may be denoted as S, where S is the set of co-phosphorylation c_{pq} values across all (p, q)∈(T∩P)×(T∩P)).

Continuing with this example, for a kinase k, Tₖ⊂T may be defined as the set of phosphosites that are reported in PhosphoSitePlus as the substrates of kinase k. For a pair of phosphosites (p, q)∈P, Pr(C_{pq}>c_{pq}|S) is the probability that the co-phosphorylation of p and q would be higher than c_{pq} given that p and q share a kinase. On the other hand, Pr(C_{pq}>c_{pq}|A) represents the probability that the co-phosphorylation of p and q would be higher than c_{pq} for any pair of phosphosites. As an example, the predictor 24 can be programmed to employ a Bayesian method and compute the log-likelihood of the association of phosphosite p with kinase k using the weights of edges between p and its neighbors that are in Tₖ, such as according to the following:

$$h ( k , p ) = {\sum }_{∀ q ∈ \{ P ⋂ {T}_{k} \}} log 2 ( \frac{Pr ( {C}_{pq} > {c}_{pq} \vert S )}{Pr ( {C}_{pq} > {c}_{pq} \vert A )} )$$

For a given phosphosite p∈P, the predictor 24 can computes all h(k,p) values for all kinases k. The predictor (or other method) 24 further can ranks the kinases in decreasing order of the score h(k,p), such that a larger value of h(k,p) indicates that k is more likely to be a kinase that phosphorylates p.

The idea behind this approach is that the likelihood of the association of a phosphosite with a given kinase or phosphatase is proportional to the fraction of its neighbors in the co-phosphorylation network that are also associated with the given kinase. Thus, the predictor 24 can be programmed to directly incorporate the dynamics of phosphorylation and/or dephosphorylation into its prediction methods. Furthermore, since the co-phosphorylation network can be context-specific, the predictor 24 method can further specify context-specific KSAs as well as context-specific PSAs. As a result, the co-phosphorylation approach implemented by the system 10 enables predictions to uncover these relationships in its relevant cellular or disease context as reflected in the original choice of MS samples and data.

As a further example, as some phosphosites can be phosphorylated by multiple kinases, ascertaining which individual kinase is activated in different cell or tissues types is challenging to predict and yet crucial to the drug discovery process. Therefore, integrating MS-based phosphoproteomic data into KSA predictions enables the KSA data 26 to capture the context-specificity of these interactions. This context-specific feature is absent from existing approaches, since sequence motifs, structural information, and protein interactions considered by these methods do not represent a specific biological context, at least with the current state of cell based information. Moreover, KSAs identified in the KSA data for a patient or groups of patients (e.g. those with a specific disease sub-type) can be used to deliver one or more therapeutic agents (e.g., a kinase inhibitor) specifically to target one or more kinases indicated in the KSA data. As one example, the therapeutic agent can be a selective kinase inhibitor, such as designed to inhibit the PRKACA (or one or more other) kinase for regulating kinase signaling pathways in the treatment cancer (e.g., lung, ovarian or breast cancer). By the same logic with respect to interactions predicted for phosphatases and phosphosites, a PSA can be predicted and a phosphatase can be targeted with a therapeutic agent. Thus, the approach disclosed herein with respect to KSAs is equally applicable to other PTMs and corresponding PTM sites and for predicting corresponding ESAs.

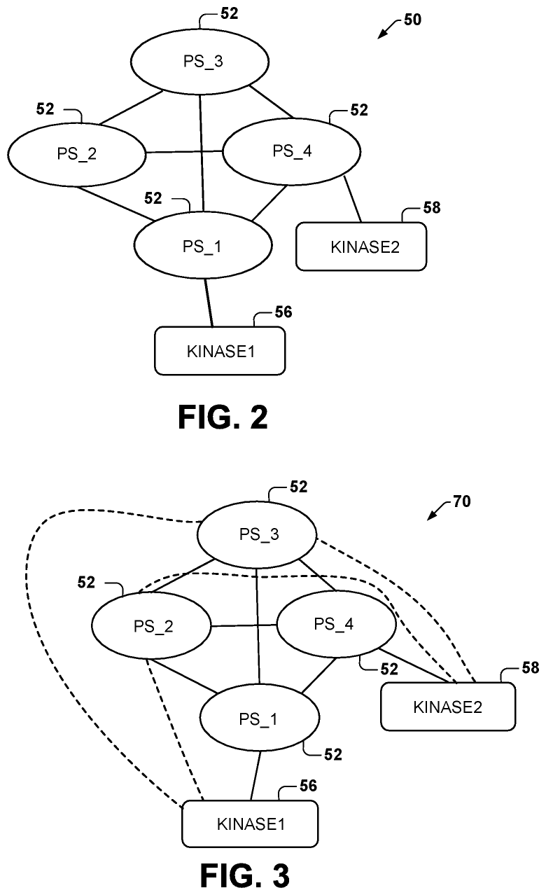

FIGS. 2 and 3 depict diagrammatic representations of co-PTM graphs 50 and 70, respectively, to help demonstrate the approach disclosed herein with respect to Kinases. In FIG. 2, the graph 50 includes a plurality of PTM site nodes 52, corresponding to phosphosite nodes demonstrated at PS_1, PS_2, PS_3 and PS_4. In the graph 50, each of the nodes 52 are interconnected by corresponding edges. As disclosed herein, each of the edges may be assigned (e.g., by network generator 22) a corresponding edge weight representing the co-phosphorylation between the respective phosphosites at the end nodes 52 connected by such edge. By way of illustration the graph 50 also includes a plurality of kinases 56 and 58, shown as KINASE1 and KINASE2. In the example network 50 of FIG. 2, one kinase (KINASE1) has a known association with phosphosite PS_1 and the other kinase (KINASE2) has a known association with phosphosite PS_4. The known KSA information can be stored in memory (e.g., as data 20), such as disclosed herein.

Based upon the co-phosphorylation among each pair of phosphosites 52, as encoded by the edge weights, a co-phosphorylation distribution can be determined (e.g., by predictor 24) to identify shared-kinase pairs of phosphosites. A shared-kinase pair of phosphosites may be identified based on analysis of known KSA data (e.g, data 20). Thus a set of corresponding edge weights that share kinases can be determined from the network 50. For example, the co-phosphorylation values (edge weights) can be compared with respect to which phosphosite is the substrate for a given kinase to transfer KSAs from the known KSA data to the phosphosites in the graph 50.

As shown in FIG. 3, a modified network 70 is illustrate after determining (e.g., by predictor 24) additional KSAs by computing a score of association between the phosphosites 52 and each of the respective kinases 56 and 58. The additional KSAs determined (e.g., by predictor 24) are demonstrated dashed lines between kinases 56 and 58 and respective phosphosites 52, as shown. The association between each of the kinases 56 and 58 and the phosphosites can be stored in memory as a KSA score (e.g., KSA data 26). The resulting KSAs further may be ranked and sorted into an order of descending rank. In other examples, a threshold (e.g, which may be fixed or user programmable) can be used to identify a set of KSAs that have scores above the threshold. Thus, the network 70 or another data structure can be constructed to represent KSAs for each of the phosphosites 52. The concepts illustrated with respect to FIGS. 2 and 3 for phosphorylation sites and kinases are equally applicable to other types of PTM sites for predicting corresponding ESAs, as disclosed herein.

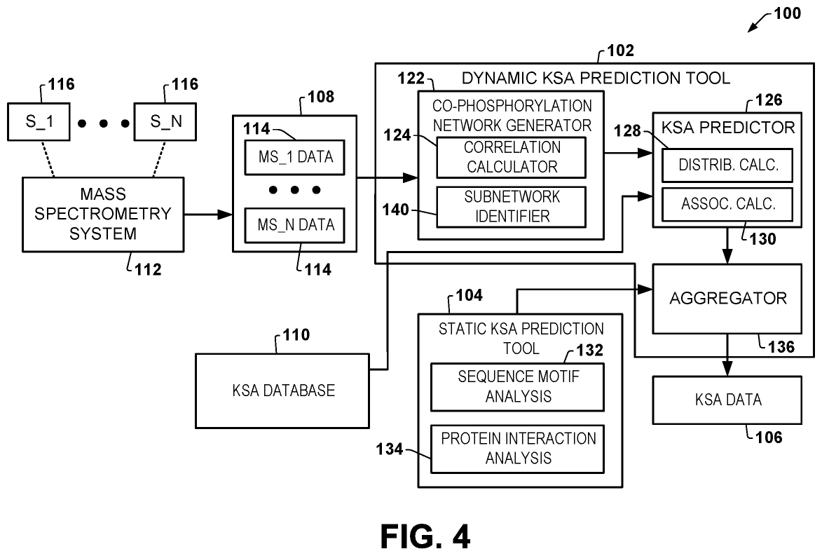

FIG. 4 depicts another example of a system 100 for identifying and quantifying KSAs. The system 100 in the example of FIG. 4, the system 100 utilizes a dynamic KSA prediction tool 102 and a static KSA prediction tool 104 and integrates the predictions from both to provide corresponding KSA predictions as corresponding KSA data 106. The dynamic tool 102 receives as inputs phosphoproteomic data 108 (e.g., corresponding to data 18) and available KSA data from one or more KSA databases 110 (e.g., corresponding to data 20). For example, the KSA data in the database 110 includes only a small percentage of KSAs that have been determined for the known phosphosites, such as for less than 10% (e.g., about 5%) of the phosphosites. Thus, the system 100 affords the ability to bridge the gap and predict the unknown KSAs and/or PSAs. In some examples, the proteomic data 108 includes data specifying phosphorylation profiles for identified phosphosites. For each phosphosite, the phosphorylation profile data may include a phosphosite identifier (e.g., a name, amino acid sequence or other identifier), data identifying each of a plurality of biological states as well as quantitative phosphoproteomic data. Each phosphosite that is provided in the proteomic data 108 thus includes a phosphorylation profile which the tool 102 uses to compute the measure of correlation between phosphosites, as disclosed herein.

As an example, the quantitative data can be provided by a mass spectrometry system 112. The mass spectrometry system 112 is configured to provide mass spectrometry data to identify phosphosites (e.g., peptide sequences) and intensity values associated with each identified phosphosite. Those skilled in the art will understand and appreciate various approaches to analyze the raw data from the mass spectrometry system 112 to assess and identify the phosphosites within a desired confidence level for each of the plurality of N biological states. For example, the mass spectrometry data includes normalized intensity mass spectrometry data (e.g., intensity values as a function of mass-to-charge (m/z)) for each of a plurality of identified phosphosites. The mass spectrometry system 112 can generate mass spectrometry data 114 for each of a plurality of N biological states, 116 demonstrated as S_1 through S_N, where N is a positive integer denoting the number of biological states. As mentioned, each biological state, for example, may correspond to an independent sample for a given biological context (e.g., tumor type or disease state). Example biological states may include a different dosage, different tumors or different time points to name a few.

The predetermined KSA data 110 may be stored in memory (e.g., local or remote) and used by the system 100. The predetermined KSA data 110 could be provided and generated by one or more KSA identification tools. For example, the database can correspond to the database provided by PhosphoSitePlus, which is available online from Cell Signaling Technology, Inc. of Danvers, Mass. Other ESA databases could be used to provide the predetermined ESA data 110 in other examples, such as the human DEPhOsphorylation database DEPOD (available online at (http://www.depod.org).

The dynamic KSA prediction tool 102 thus receives as inputs the proteomic data 108 and the predetermined KSA data 110. In describing example pseudocode for the methods of the dynamic KSA prediction tool 102, the inputs and outputs may correspond to the following:

Input:

(P, B, M ): Phosphoproteomics data

P : phosphosites (|P | = m)
B: biological states (|B| = n)
M ∈ R^{m}*ⁿ : quantitative phosphoproteomics
Mi ∈ R^{l}*ⁿ is the phosphorylation profile

(T, K): PhosphoSitePlus data

T: substrates
K: kinases (|K| = z)
Ti is the substrate of a kinase Ki

Output:

H ∈ R^{m}*^{z} : kinase-substrate association scores by dynamic tool
102

H(p, k) represents the association of kinase k to phosphosite
p

The dynamic KSA prediction tool 102 includes a co-phosphorylation network generator 122. The network generator 122 constructs a co-phosphorylation network data structure that includes a node for each of the phosphosites identified in the input data 108. The network also includes edges between all pairs of phosphosite nodes to form a complete graph. The network generator 122 also includes a correlation calculator 124 to compute edge weights to each of the edges that are connected between respective pairs of phosphosite nodes. As one example, the correlation calculator 124 is programmed to compute a biweight midcorrelation between each pair of phosphosite nodes. The network generator 122 in turn produces the co-phosphorylation network (e.g., a graph) including the phosphosite nodes and weighted edges defining the co-phosphorylation between each pair of nodes. The co-phosphorylation network is provided to a KSA predictor 126. An example of pseudo code corresponding to the network generator 122 is demonstrated below.

| [symbol] Constructing co-phosphorylation network G =< V, E > | |
|---|---|
| [symbol] Nodes V: phosphosites P | |
| [symbol] Edges E: between all pairs of P (complete graph) | |
| C ← null | [symbol] A set of edge weights of the co-phosphorylation |

network

for p ∈ P do

for q ∈ P do

c_{pq} = biweight-midcorrelation(Mₚ, M_{q} )
C ← C ∪ c_{pq}

end for

end for

The KSA predictor 126 is configured to compute KSA scores representing the association of each kinase to one of the respective phosphosites in the network generated by network generator 122. The KSA predictor 126 includes a distribution calculator 128 programmed to determine a co-phosphorylation relation distribution of shared-kinase pairs of phosphosites in the co-phosphorylation network. The distribution calculator 128 determines the distribution based on the predetermined KSA data 110 and the co-phosphorylation network. The distribution calculator 128 thus identifies which pairs of phosphosites share a kinase according to the predetermined KSA data 110.

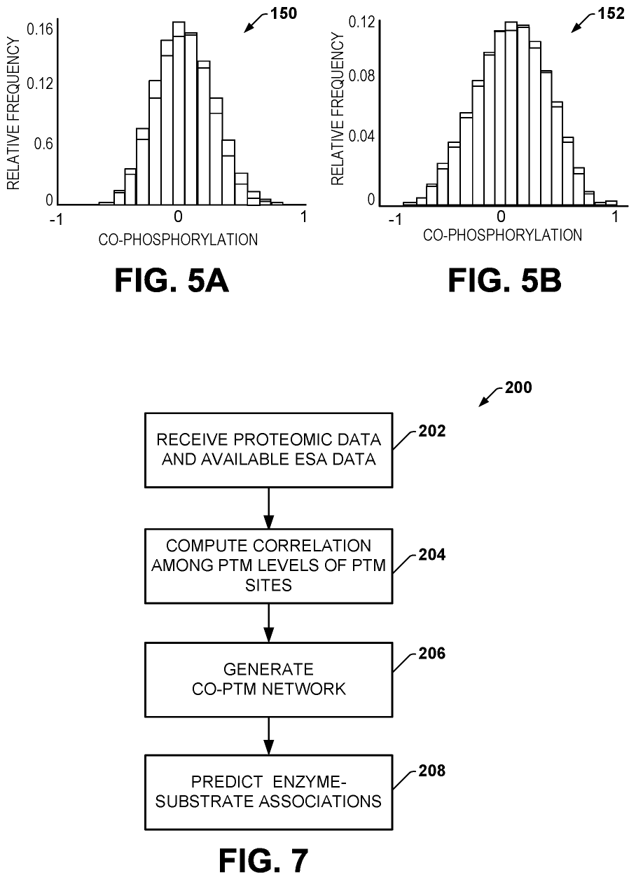

FIGS. 5A and 5B demonstrate examples of distributions 150 and 152, respectively, which may be computed by distribution calculator 128. The distributions 150 and 152 show relative frequency of co-phosphorylation of shared-kinase pairs as compared to all other phosphosite pairs.

The example distribution 150 is determined by distribution calculator 128 from a Breast Cancer PDX dataset (e.g., 37234 shared kinase pairs; μ=0.05, σ=0.23, kutosis=2.85, skewness=0.10). The example distribution 152 is determined by distribution calculator 128 for Ovarian Cancer tumors (e.g., 8235 shared kinase pairs; μ=0.11, σ=0.32, kutosis=2.54, skewness=−0.10). As shown in FIGS. 5A and 5B, for both datasets, these two distributions 150 and 152 are significantly different and the co-phosphorylation distribution of shared-kinase pairs is shifted to the right (e.g., Kolmogorov-Smirnov test p-value respective <7.36E-103, 1.1E-6 for breast cancer PDX and ovarian cancer, respectively). In other words, the distributions 150 and 152 demonstrate that substrates that share a kinase are more likely to be positively co-phosphorylated compared to all pairs of phosphosites.

Although this positive correlation of substrate pairs can be potentially explained by shared kinase annotation, the data in distributions 150 and 152 also contains negative correlations of phosphosite pairs that point to more complex regulatory relationships. One reason for this observation could be that for some phosphosites, multiple kinases have been reported in the predetermined KSA data 110, but the annotation does not provide context-specific information, e.g., the kinases might not all be active at the same time.

An example of pseudo code corresponding to the distribution calculator 128 is shown below.

[symbol]

Find co-phosphorylation distribution of shared-kinase pairs of

phosphosites

S ← null [symbol] A set of edge weights of pairs of phosphosites that

share the kinase according to PSP

for t ∈ T do

for r ∈ T do

| | |
|---|---|
| if (t, r) ∈ E then | [symbol] both substrates exist in the |

network/experiment

S ← S ∪ ctr

end for

end for

Referring back to FIG. 4, the KSA predictor 126 also includes an association calculator 130. The association calculator 130 is programmed to compute a score for association between each of the phosphosites identified in the proteomic data 108 and all known kinases. The predetermined KSA data 110 thus provides a reference for known KSAs that are utilized by the association calculator 130 for transferring the association from the known KSAs to the phosphosites in the co-phosphorylation network being analyzed by the association calculator 130.

As an example, the association calculator can employ a naïve Bayesian method to calculate the probability of a KSA association of a given kinase to each of the phosphosites, such as described with respect to predict 24 of FIG. 1. Thus, the association calculator 130 uses the computed co-phosphorylation between phosphosites (e.g., determined by calculator 124 of network generator 122) to ascertain associations of those phosphosites with the given kinase. The KSA predictor 126 in turn outputs the association score (e.g., a log likelihood score) between each of the phosphosites (specified in data 108) and kinases.

An example of pseudo code that can be utilized to implement the association calculator 130 is shown below.

| | |
|---|---|
| [symbol] | Compute score of association between phosphosites and kinases |

H ← 0
for p ∈ P do
for k ∈ K do

| | |
|---|---|
| Subk ← {Ti\|Ki = k} | [symbol] Substrates of kinase k |

for x ∈ Subk do

| | |
|---|---|
| if (p, x) ∈ E then | [symbol] Substrate x exists in the network |

```

[symbol]
      Naive Bayesian Rule
      $H ( p , k ) = H ( P , k ) + {log}_{2} ( \frac{\vert s > {c}_{px} \vert}{\vert s \vert} / \frac{\vert C > {c}_{px} \vert}{\vert C \vert} )$
    end for
  end for
end for
sort each row of H in descending order
return H
```

In this way, the corresponding KSAs can be ascertained for each of the phosphosites. In addition to using KSAs produced by the dynamic KSA prediction tool 102, the system 100 may also integrate one or more static KSA prediction tools, demonstrated at 104. In this example, the KSA prediction tool 104 includes sequence motif analysis 132 and protein interaction analysis 134. In other examples, one or more other tools or combinations of analysis may be used to predict KSAs.

By way of an example, the static tool 104 may correspond to KinomeXplorer (available online at http://kinomexplorer.info) such that the sequence motif analysis 132 may correspond to NetworKIN and the protein interaction analysis 134 may correspond to NetPhorest. The static KSA prediction tool of the system 100 is demonstrated as including the static KSA prediction tool 104, it is understood that the dynamic tool 102 may include an interface to access the tool 104 at a remote resource location (e.g., at a uniform resource locator) and communicate the corresponding phosphosite data as inputs to each of the analysis functions 132 and 134. The static KSA prediction tool 104 thus provides a score reporting the likelihood of KSAs.

An aggregator 136 can be programmed to combine the KSA scores from each of the prediction tools 102 and 104 and provide the KSA data 106 as an aggregate KSA score for some or all phosphosites. In some examples, such as where the association calculator 130 computes to be KSAs as a log likelihood, the aggregator function 136 may compute the log of the score provided by the tool 104 so that the association scores are on a common scale prior to being summed by the aggregator for each of the phosphosites. The aggregator 136 in turn provides the combined KSA data 106 which can be stored in memory (e.g., 12).

As a further example, assume that the score for produced by tool 104 for the interaction between kinase k to phosphosite p is x(k,p). The aggregator can compute the aggregate score (i.e. M(k,p)) for phosphosite p and kinase k by combining scores as follows:

M(k,p)=h(k,p)+log 2(x(k,p))

As in the example system 100 of FIG. 1, the system 100 also computes M(k,p) for all phosphosite-kinase pairs. To facilitate evaluation and kinase identification, the aggregator or another function may be programmed to rank kinases for each phosphosite, such that a larger value of M(k,p) indicates that k is more likely to be a kinase that phosphorylates p. An example of the algorithm that may be implemented to compute the combined dynamic and static score M(k, p) is as follows:

Input:

H ∈ R^{m}*^{z} : Scores from dynamic tool (102)
N ∈ R^{f}*^{b} : Scores from static tool (104) for W phosphosites over X
kinases,
|W|=f,|X|=b

Output:

F ∈ R^{m}*^{z}: Aggregate kinase-substrate association scores by system
(100)

F ← 0
for p ∈ P do

for k ∈ K do

| | |
|---|---|
| if p ∈ W & k ∈ X then | [symbol] the phosphosite and |

kinase has association score in static tool

F(p, k) = H(p, k) + log(N(p, k))

else

F(p, k) = H(p, k)

end for

end for
sort each row of F in descending order
return F

While the example of FIG. 4 describes the system 100 for predicting KSAs, the system may, additionally or alternatively, be adapted to identify and quantify other ESAs.

In some examples, the system 100 may also include a subnetwork identifier 140, which is demonstrated as part of the network generator. In one example, the subnetwork identifier 140 is programmed to configure each of two or more subnetworks that define the co-phosphorylation network based on different sets of phosphorylation profiles. For example, the co-phosphorylation (e.g., determined by correlation calculator 124) between subtypes is used to find an active pathways that are associated with the subtype. The result is consistence with the known subtype-associated pathways. Thus, each of the subnets and respective phosphorylation profiles may be subtype specific such that the extracted subnetworks likewise include phosphosites associated with subtype-specific pathways.

In another example, the subnetwork identifier 140 is programmed to generate a co-phosphorylation network of physically interacting proteins (PPI weighted by co-phosphorylation). For example, the subnetwork identifier can receive an input of protein-protein interactions (PPI), such as from the protein interaction analysis tool 134. The subnetwork identifier 140 can provide the co-phosphorylation network to includes two or more subnetworks of physically interacting proteins in which the associations between proteins are weighted by the co-phosphorylation (e.g., computed by correlation calculator 124), as disclosed herein.

By way of further example, KSA prediction tool 102 or both tools 102 and 104 may use large scale phosphoproteomics data obtained from multiple samples that represent different cancer subtypes, e.g., luminal and basal breast cancer. The data can be generated to analyze the effect of delayed cold Ischemia on phosphoproteins' stability in tumor samples using quantitative LC-MS/MS. The phosphorylation changes were calculated relative to the phosphorylation level of proteins of the tumor samples that were frozen immediately with a time span of less than 1 minute from excision to freezing in liquid nitrogen. The phosphorylation level of 3 tumors of each subtype across 3 time points (i.e. 9 dimensions for each subtype) are used to assess the co-phosphorylation of pairs of phosphosites, where co-phosphorylation is defined as the correlation of phosphorylation levels across different cellular states. Protein phosphorylation of nearly 10000 phosphosites corresponding to ˜3000 proteins thus may be used for further analysis.

As an example, two example protein-protein interaction (PPI) networks were constructed (e.g., using BioGRID) such that the weight of the interactions are the co-phosphorylation between two interacting proteins in basal and luminal. To address the challenge of identifying subtype-specific modules, for example, Modularity-based Scoring Schema (MoBaS) may be used. To assess the statistical significance of the identified subnetworks, the phosphoproteomics data may be permuted. Permutation breaks the relationship between phosphosites and effect the co-phosphorylation. As a result, the topology of the network is still the same, and just the weights are randomized/permuted. That is, in each PPI network that is constructed, the topology of the networks that are used for different subtypes are similar as it is protein-protein interaction network. However, the edge weights are subtype-specific because the phosphorylation profile of each phosphosite comes from different subtype data.

As further example, assuming there are 9 luminal tumors and 9 basal tumors, each phosphosite has two phosphorylation profiles (one for each context): (i) a 9-dimension vector from luminal data (ii) a 9-dimension vector from basal data. Using two sets of phosphorylation profiles, two weighted networks may be constructed. MoBaS uses the co-phosphorylation values to extract the subnetworks from a huge weighted PPI network. Pathway enrichment analysis on the extracted subnetworks show the extracted subnetworks from luminal network are enriched in the pathways that are known to be associated with luminal subtype and the extracted subnetworks from basal network are enriched in the known pathways that are associated with basal subtype. It can also be shown that in the pathways that are known to be associated to breast cancer, there are some phosphosites that are positively correlated in one subtype, and negatively correlated in another subtype. This indicates that in the breast cancer pathways, the phosphorylation might be rewired such that the subtypes can be distinguished.

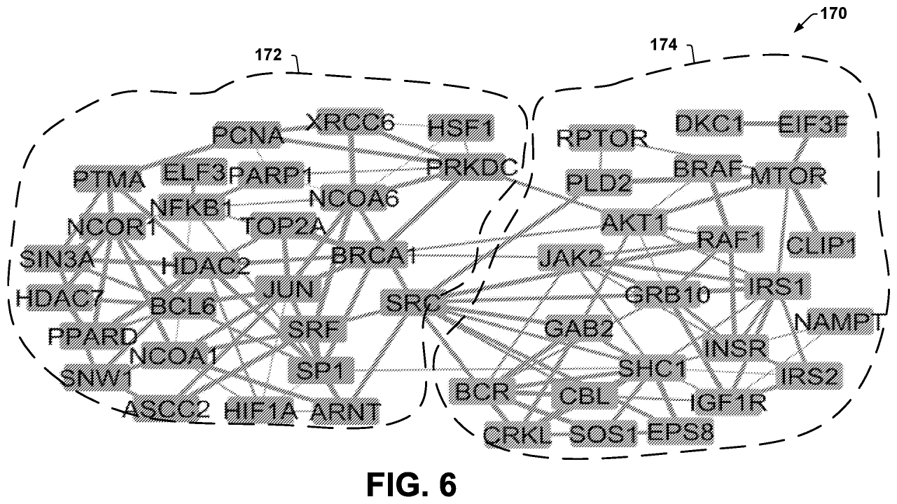

FIG. 6 depicts an example of a PPI network 170 that includes subnets 172 and 174, such as generated by subnetwork identifier 140. In the example, of FIG. 6, the associations between nodes, as represented by edges, are demonstrated as having a line thickness that is set as a function of the co-phosphorylation between respective proteins. In other examples, the co-phosphorylation may be visualized by different graphical features (e.g., line color, line length or text values of co-phosphorylation or the like). A description of the active pathways associated with each subnet 172 and 174 are described in the following table. Additional subnets associated with different pathways may be constructed in other examples.

| | |
|---|---|
| Pathway | P-value |

Subnet1 (172)

| | |
|---|---|
| Thyroid hormone signaling pathway | 3.7E−7 |
| Apoptosis | 0.005 |
| Notch signaling pathway | 0.009 |
| MAPK signaling pathway | 0.01 |
| ErbB signaling pathway | 0.01 |
| Wnt signaling pathway | 0.03 |

Subnet2 (174)

| | |
|---|---|
| EGFR signaling pathway | 5.7E−20 |
| Kit receptor signaling pathway | 2.5E−16 |
| MAPK signaling pathway | 2.3E−5 |
| ErbB signaling pathway | 1.48E−12 |

The integration of protein interaction network and proteomics data, as disclosed herein, can help to infer active pathways that are associated with different subtypes. Additionally, it can be shown that the identified functional modules are reproducible across two independent datasets. Analysis further reveals rewiring of co-phosphorylation network in different subtypes. These rewiring might also affect pathways that are associated with certain disease states (e.g., cancers). These results imply that the patterns of phosphorylation (i.e. co-phosphorylation) can provide insight in distinguishing subtypes and further can be used in development of subtype-specific therapeutic agents, for example.

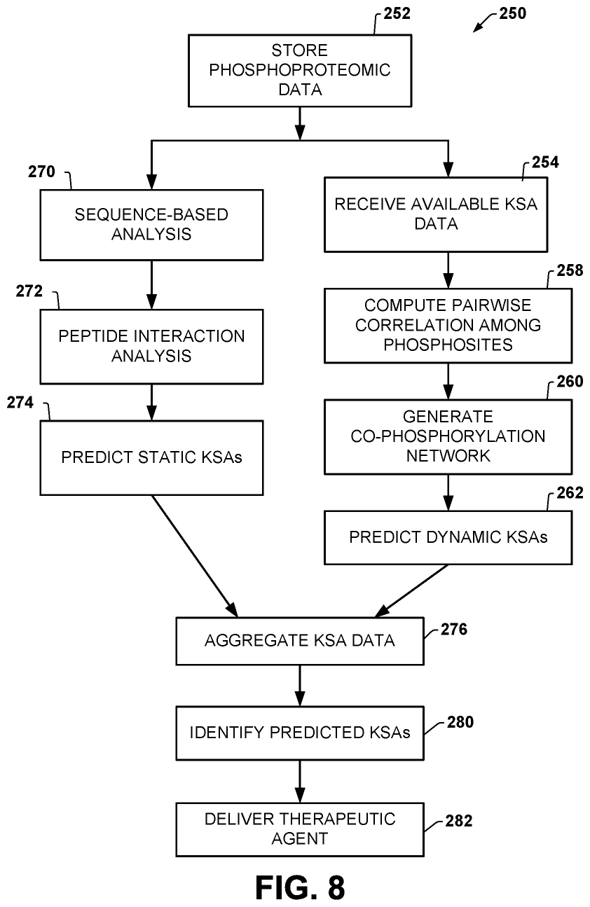

In view of the foregoing structural and functional features described above, example methods will be better appreciated with reference to FIGS. 7 and 8. While, for purposes of simplicity of explanation, the example methods of FIGS. 7 and 8 are shown and described as executing serially, it is to be understood and appreciated that the present examples are not limited by the illustrated order, as some actions could in other examples occur in different orders, multiple times and/or concurrently from that shown and described herein. Moreover, it is not necessary that all described actions be performed to implement a method. The example method of FIGS. 7 and 8 can be implemented as instructions and data stored in one or more machine readable media (e.g., program code). The stored instructions can be executed by a processing resource (e.g., one or more processor cores) and executed to perform the methods disclosed herein.

FIG. 7 depicts an example of a method 200 that can be utilized to predict ESAs. The method begins at 202 in which context-specific proteomic data and available ESA data are received as inputs (e.g., corresponding to data 18, 20, 108 and 110). At 204, a correlation among the PTM levels of PTM sites is computed (e.g., by calculator 16, 124) from phosphoproteomic data to provide a co-PTM measure. The correlation, for example can be computed as a pairwise biweight midcorrelation or another measure of correlation for each pair of PTM sites in the data can be used.

At 206, a co-PTM network is generated (e.g., by network generator 22, 122). The network is generated based on the identified PTM sites in the data received at 202 and the computed correlation at 204. The network thus includes nodes corresponding to each of the respective PTM sites and edges between pairs of nodes having weights assigned to represent the computed co-PTM between such nodes. For the example of phosphorylation, the network is a co-phosphorylation network that includes nodes corresponding to each of the respective phosphosites and edges between pairs of nodes having weights assigned to represent the computed measure of co-phosphorylation.

At 208, a set of ESAs are identified (e.g., by predictor 24, 126). The ESAs may be predicted by analyzing distributions of shared-enzyme pairs for PTMs and computing association scores for PTM sites and all enzymes. As one example, where the ESAs are KSAs, the KSAs can be identified based on computing (e.g., by distribution calculator 128) distributions of shared-kinase pairs of phosphosites and then computing (e.g., by association calculator 130) an association score for phosphosites and all kinases. As another example, where the ESAs are PSAs, the PSAs can be identified based on computing (e.g., by distribution calculator 128) distributions of shared-phosphatase pairs of phosphosites and then computing (e.g., by association calculator 130) an association score for phosphosites and all phosphatases. For example, the association score for each PTM site may be computed by implementing (e.g., in predictor 24, 126) a Bayesian method, such as a naïve Bayesian method, as disclosed herein. Based on one or more ESAs identified in ESA data provided at 208, therapeutic agent(s) may be administered to a patient. For example, the therapeutic agent may include a kinase inhibitor (or phosphatase activator, to mediate the same effect) known to inhibit (or modulate up as indicated with a phosphatase activator) one or more of the identified ESAs (e.g, KSAs or PSAs).

FIG. 8 is a flow diagram depicting another example method 250 that may be used to identify KSAs. The method 250 may be considered as having multiple prediction paths that may operate in parallel, one providing prediction on static information and the other being more global and based on dynamic, context specific information from mass spectrometry and/or similar data. For purposes of ease of explanation, the method 250 is described with respect to predicting KSAs. As disclosed herein and similar to the example of FIG. 7, however, the method 250 is equally applicable to, additionally or alternatively, predict other ESAs including PSAs.

The method 250 begins at 252 in which phosphoproteomic data is received as an input (e.g., corresponding to data 18, 108). For example, the phosphoproteomic data provides a phosphorylation profile for each identified phosphosite. Each phosphorylation profile may enumerate a set of identified phosphosites, biological states and quantitative phosphoproteomic for the identified phosphosites. Based on the input data, the method 250 may implement multiple (parallel) processing paths, which in some examples may be selectively activated (e.g., in response to a user input). The different processing paths may be implemented concurrently or sequentially. Thus from 252, the method proceeds to 254 for implementing dynamic KSA prediction (e.g., corresponding to dynamic prediction tool 102) and to 270 for implementing static KSA prediction (e.g., corresponding to static prediction tool 104 and/or method 200).

At 254, predetermined KSA data is stored. This can be implemented as part of receiving input data in conjunction with the phosphoproteomic data at 252 or separate as shown. The predetermined KSA data may describe available KSAs, such as may be provided by another tool or one or more databases that are accessible to view known KSAs.

At 258, a pairwise correlation among the phosphosites is computed (e.g., by calculator 16, 124) from phosphoproteomic data (received as input data at 252) to provide a co-phosphorylation measure. For example, the correlation can be computed as a biweight midcorrelation of normalized intensity mass spectrometry data or another measure of correlation for each pair of phosphosites can be used. In another example, the correlation may be computed to represent co-phosphorylation of the phosphorylation profiles associated with each respective phosphosite pair.

At 260, a co-phosphorylation network is generated (e.g., by network generator 22, 122) for the identified phosphosites based on the computed correlation at 258. For example, the network is a graph data structure that includes nodes representing each of the respective phosphosites linked by weighted edges to represent the computed co-phosphorylation between each pair of phosphosites.

At 262, a set of dynamic KSAs are predicted (e.g., by predictor 24, 126). The KSAs can be predicted (e.g., by distribution calculator 128) by computing distributions of shared-kinase pairs of phosphosites and then computing (e.g., by association calculator 130) an association score for phosphosites and all kinases based on the distributions. For example, the association score may be computed by implementing (e.g., in predictor 24, 126) a Bayesian method, such as disclosed herein.

With reference to the other processing path, at 270, sequence based analysis is performed on the phosphoproteomic data. For example, the phosphosites (e.g., an amino acid sequence) can be input to a tool (e.g., NetPhorest available at http://netphorest.info/index.shtml) programmed to analyze patterns in sequence motifs and thereby predict KSAs. At 272, peptide interaction analysis is performed by a corresponding tool (e.g., NetworKIN available at http://networkin.info/index.shtml) based on the phosphosites identified in the phosphoproteomic data. The analysis at 272 returns a set of KSA predictions. The tools configured to perform the analyses at 270 and 272 may be part of an integrated framework for modeling kinase-substrate associations (KinomeXplorer available at http://kinomexplorer.info/). Additional information about the analysis and predictions at 270 and 272 are described in Horn H, et al. “KinomeXplorer: an integrated platform for kinome biology studies”, Nat. Methods, vol. 11, pg. 603-604 (May 2014)). Additional or alternative tools for performing the analyses may be used in other examples. At 274, the KSAs predicted by the analyses are provided (e.g., as predicted KSA data) and stored in memory.

At 276 the KSA data provided at 274 and 262 are aggregated. For example, the data at 274 may provide KSA annotations for about 30%-40% of the identified phosphosites, whereas the data produced by the dynamic path at 262 annotates all the identified phosphosites. Additionally, by aggregating the predictions at 276 the accuracy of the annotations provided at 274 will be improved. Any differences between the format of the data set may be resolved to enable the KSA predictions to be combined on a common scale. At 280, a dataset of predicted KSAs for all identified phosphosites is provided. The scores associated with each of the predicted KSAs may be used to rank and sort the KSAs. In some examples, due to the context specificity of the phosphoproteomic data, the output may include a list target kinase rankings for a given context (e.g., a disease). The rankings of target kinases may be derived from statistical analysis of KSAs predicted in each of the prediction methods or from the combined data set.

At 282, a therapeutic agent is delivered to a patient based on the predicted KSAs at 280. For example, the therapeutic agent (e.g., a kinase inhibitor) can be designed to inhibit one or more kinases to treat a disease (e.g., cancer), such as disclosed herein. In some examples, the target ranking of kinases from the predicted KSA data (at 280) may be used as input to one or more databases to identify one or more known therapeutic agents having properties to inhibit or otherwise regulate the identified kinase(s).

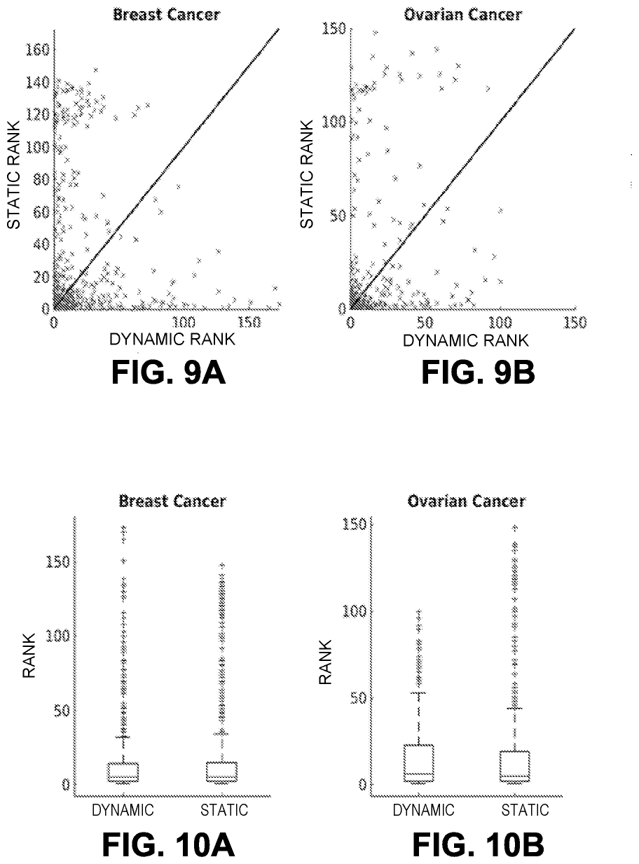

By way of example, FIGS. 9A and 9B depict graphs comparing rankings for KSAs predicted via each of the static processing path (on the y-axis) and dynamic processing path (on the x-axis for different biological contexts, namely breast cancer and ovarian cancer, respectively. In particular, FIG. 9A includes rankings of KSA prediction for 313 kinase-substrate association predictions for the breast cancer PDX data. FIG. 9B includes rankings of KSA predictions for 740 kinase-substrate association predictions for the ovarian cancer data. In FIGS. 9A and 9B, a point that is closer to the origin indicates higher ranking. If the two prediction methods were consistently correct in their predictions, the graphs of FIGS. 9A and 9B would include a cluster of points only around the origin. In fact aside the dense cluster around the origin, many predictions are “close to the lines”, indicating a high rank from one approach and a low rank from the competing method. This suggests that these two methods contribute different information; therefore integrating the prediction of these two methods might improve the predictions.

FIGS. 10A and 10B show the same data as FIGS. 9A and 9B visualized in box plot format. That is, FIGS. 10A and 10B show the box plot distributions of target kinase rankings for the breast cancer PDX and ovarian cancer data. This visualization format further demonstrates that the distribution of results is overall similar.

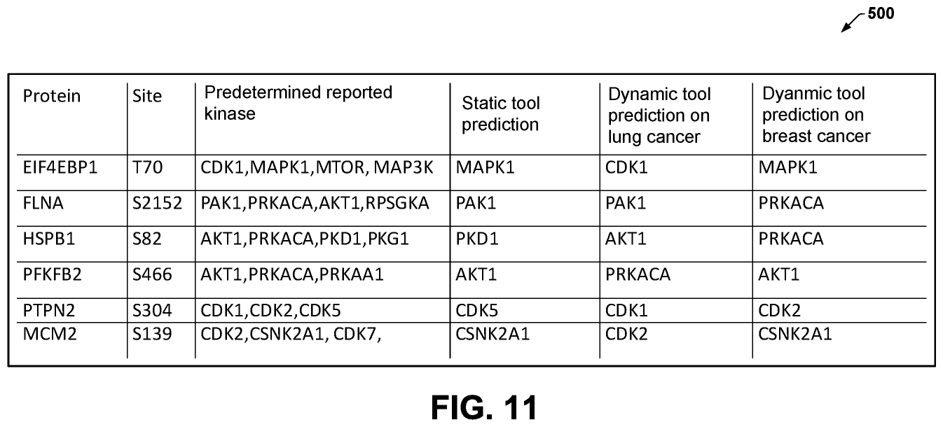

FIG. 11 depicts a table 300 demonstrating data-specific kinase predictions that can be provided based on the systems and methods disclosed herein for different biological contexts, namely lung cancer and breast cancer. The phosphosites listed in this table are reported to have more than one predetermined kinase (e.g, as provided by PhosphoSitePlus). The systems and methods disclosed herein (under columns titled “dynamic tool prediction”) include an identification of kinases previously reported, but also identify different kinases as the top-ranked candidate based on each dataset compared to the static tool prediction, which are not context specific.

In view of the foregoing structural and functional description, those skilled in the art will appreciate that portions of the invention may be embodied as a method, data processing system, or computer program product. Accordingly, these portions of the invention may take the form of an entirely hardware embodiment, an entirely software embodiment, or an embodiment combining software and hardware. Furthermore, portions of the invention may be a computer program product on a computer-usable storage medium having computer readable program code on the medium. Any suitable computer-readable medium may be utilized including, but not limited to, static and dynamic storage devices, hard disks, optical storage devices, and magnetic storage devices.

Certain embodiments of the invention have also been described herein with reference to block illustrations of methods, systems, and computer program products. It will be understood that blocks of the illustrations, and combinations of blocks in the illustrations, can be implemented by computer-executable instructions. These computer-executable instructions may be provided to one or more processor of a general purpose computer, special purpose computer (e.g., as part of the imaging system), or other programmable data processing apparatus (or a combination of devices and circuits) to produce a machine, such that the instructions, which execute via the processor, implement the functions specified in the block or blocks.

These computer-executable instructions may also be stored in computer-readable memory that can direct a computer or other programmable data processing apparatus to function in a particular manner, such that the instructions stored in the computer-readable memory result in an article of manufacture including instructions which implement the function specified in the flowchart block or blocks. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions specified in the flowchart block or blocks.

What have been described above are examples. It is, of course, not possible to describe every conceivable combination of components or methodologies, but one of ordinary skill in the art will recognize that many further combinations and permutations are possible. Accordingly, the disclosure is intended to embrace all such alterations, modifications, and variations that fall within the scope of this application, including the appended claims. As used herein, the term “includes” means includes but not limited to, the term “including” means including but not limited to. The term “based on” means based at least in part on. Additionally, where the disclosure or claims recite “a,” “an,” “a first,” or “another” element, or the equivalent thereof, it should be interpreted to include one or more than one such element, neither requiring nor excluding two or more such elements.

1. A system, comprising:
one or more non-transitory machine readable media to store instructions executable by a processor to perform a method, the instructions comprising:
correlation calculator code to compute a co-post-translational modification (PTM) value based on a measure of correlation of intensity data for pairs of PTM sites identified in context-specific proteomic data;
network generator code to construct a co-PTM network for the PTM sites identified in the context-specific proteomic data based on the co-PTM values; and
predictor code to identify enzyme-substrate associations based on analyzing the co-PTM network with respect to a set of predetermined enzyme-substrate associations.

2. The system of claim 1, wherein the context-specific proteomic data comprises mass spectrometry data generated for a number of biological states for a plurality of PTM sites identified in the mass spectrometry data, the correlation calculator code to compute a co-PTM value based on a measure of correlation of intensity data for pairs of PTM sites identified in mass spectrometry data.

3. (canceled)

4. The system of claim 2, wherein the mass spectrometry data further comprises PTM profiles associated with each of the PTM sites for each of the biological states,
wherein the correlation calculator computes the co-PTM values to represent co-PTM of the PTM profiles associated with each respective pair of the PTM sites, and
wherein the network generator is programmed to generate the co-PTM network as a graph of nodes, corresponding to each of the PTM sites identified in the mass spectrometry data, each node connected by an edge, the network generator to assign an edge weight to the edge between each pair of nodes in the co-PTM network based on the respective co-PTM value.

5. The system of claim 1, wherein the context-specific proteomic data comprises mass spectrometry data representing a number of biological states having a plurality of biological contexts, the predictor code determining context-specific kinase-substrate associations and/or context-specific phosphatase-substrate associations for each of the plurality of biological contexts.

6. The system of claim 1, wherein the correlation calculator code further computes the co-PTM value for each pair of PTM sites according to a biweight midcorrelation function.

7. The system of claim 1, wherein the correlation calculator code is programmed to compute the co-PTM value to include positive correlations and negative correlations for each of the pairs of PTM sites, the predictor code to identify the enzyme-substrate associations based on the positive correlations and/or the negative correlations.

8. The system of claim 1, wherein the enzyme-substrate associations include likelihood values for dynamic enzyme-substrate associations, the instructions further comprise:
static prediction code to derive likelihood values for static enzyme-substrate associations for a proper subset of the dynamic enzyme-substrate associations; and
aggregator code to combine the likelihood values for the dynamic enzyme-substrate associations and likelihood values for the static enzyme-substrate associations.

9. The system of claim 8, wherein the instructions further comprise code to rank the dynamic enzyme-substrate associations and to rank the static enzyme-substrate associations.

10. The system of claim 1, wherein the predictor code further comprises code to determine a distribution of co-PTM of shared-enzyme pairs between the pairs of PTM sites based on the predetermined enzyme-substrate associations.

11. (canceled)

12. The system of claim 1,
wherein the co-PTM value is computed as a co-phosphorylation value based on a measure of correlation of intensity data for pairs of phosphorylation sites identified in the context-specific proteomic data,
wherein the co-PTM network comprises a co-phosphorylation network for the phosphorylation sites identified in the context-specific proteomic data based on the co-phosphorylation values, and
wherein the enzyme-substrate associations comprise kinase-substrate associations and/or phosphatase substrate associations predicted based on analyzing the co-phosphorylation network with respect to the set of predetermined enzyme-substrate associations.

13. (canceled)

14. A method, comprising:
accessing, by a processor, proteomic data representing intensity values for each of a plurality of identified post-translational modification (PTM) sites;
computing, by the processor, a pairwise correlation of co-PTM among each respective pair of the identified PTM sites;
generating, by the processor, a co-PTM network based on each pairwise correlation to describe co-PTM characteristics for the identified PTM sites; and
predicting, by the processor, enzyme-substrate associations for each of the plurality of identified PTM sites based on the co-PTM network and a set of predetermined enzyme-substrate associations.

15. The method of claim 14, wherein the proteomic data comprises quantitative phosphoproteomic data for a number of independent biological states for each of the identified the PTM sites.

16. The method of claim 15, wherein the phosphoproteomic data further comprises PTM profiles associated with each of the PTM sites for each of the biological states,
wherein the pairwise correlation is computed to provide a measure of co-PTM of the PTM profiles associated with each respective pair of the PTM sites, and
wherein the co-PTM network is generated as a graph data structure that includes nodes, corresponding to each of the identified the PTM sites, each node connected to each other node by a respective edge,
the method further comprising assigning an edge weight to the edge between each pair of nodes in the co-PTM network based on the respective co-PTM value.

17. The method of claim 14, wherein the proteomic data further comprises the phosphoproteomic data representing a number of biological states for each of a plurality of biological contexts, the method further comprising determining a set of context-specific enzyme-substrate associations for each of the PTM sites in each of the plurality of biological contexts.

18. The method of claim 14, wherein the co-PTM network is generated as a graph data structure that includes nodes, corresponding to each of the identified the PTM sites, each node connected to each other node by a respective edge, and
wherein computing the pairwise correlation further comprises computing a co-PTM value for each respective pair of the identified PTM sites according to a biweight midcorrelation function.

19. The method of claim 14, wherein computing the pairwise correlation further comprises computing positive correlations and negative correlations for each respective pair of the identified PTM sites, the enzyme-substrate associations being predicted based on the positive correlations and/or the negative correlations.

20. The method of claim 14, wherein the enzyme-substrate associations include likelihood values for a set of dynamic enzyme-substrate associations, the method further comprising:
predicting another set of enzyme-substrate associations for a proper subset of the dynamic enzyme-substrate associations based on static information; and
aggregating each set of the of predicted enzyme-substrate associations.

21. The method of claim 14, further comprising determining a distribution of co-PTM of shared-enzyme pairs between each respective pair of the PTM sites based on the predetermined enzyme-substrate associations.

22. The method of claim 14, further comprising administering a therapeutic agent that is selected based the predicted enzyme-substrate associations.

23. The method of claim 14,
wherein the identified PTM sites are phosphorylation sites,
wherein the correlation of co-PTM is computed as a correlation of co-phosphorylation among each respective pair of the phosphorylation sites;
wherein the co-PTM network is a co-phosphorylation network describing co-phosphorylation characteristics for the identified phosphorylation sites, and
wherein the enzyme-substrate associations include kinase-substrate associations and/or phosphatase-substrate associations based on the co-phosphorylation network and the set of predetermined enzyme-substrate associations.

24. (canceled)

25. (canceled)
